(19) United States
(12) Patent Application Publication
(10) Pub. No.: US 2010/0121221 A1
(43) Pub. Date: May 13, 2010

(54) MEASURING DEVICE AND METHOD OF USING THE DEVICE TO MEASURE CONCENTRATION OF INTRAGASTRIC CONTENTS

(75) Inventor: Yuan-Soon HO, Taipei County (TW)

Correspondence Address: EGBERT LAW OFFICES, 412 MAIN STREET, 7TH FLOOR, HOUSTON, TX, 77002, US

(73) Assignee: SAND COUNTY BIOTECHNOLOGY, INC., Hsin Chu Industrial Park (TW)

(21) Appl. No.: 12/347,215

(22) Filed: Dec. 31, 2008

(30) Foreign Application Priority Data
Nov. 7, 2008 (TW) 097142987

(51) Int. Cl. A61B 5/00; G01N 27/26

(52) U.S. Cl. 600/582; 204/403.01; 600/300

(57) ABSTRACT

The present invention provides a device and method for measuring the concentration of intragastric contents. The device includes an electro-chemical specimen and a detector. The electro-chemical specimen contains a board type insulating substrate with an insertion end and sensing end. There is an electrode unit, containing a detection zone and reaction zone, of which the detection zone corresponds to the insertion end of the board type insulating substrate, whilst the reaction zone corresponds to the sensing end of the board type insulating substrate. A reaction portion, is assembled onto the sensing end of the board type insulating substrate and aligned with the reaction zone of the electrode unit. The detector includes a measurement slot, a voltage generation unit, a detection processing unit, and a measurement display screen. The device directly measures the concentrations of saccharide, protein and amino acid, etc, in the intragastric contents.

### CROSS-REFERENCE TO RELATED U.S. APPLICATIONS

Not applicable.

### STATEMENT REGARDING FEDERALLY SPONSORED RESEARCH OR DEVELOPMENT

Not applicable.

### NAMES OF PARTIES TO A JOINT RESEARCH AGREEMENT

Not applicable.

### REFERENCE TO AN APPENDIX SUBMITTED ON COMPACT DISC

Not applicable.

### BACKGROUND OF THE INVENTION

1. Field of the Invention

The present invention relates generally to a measuring device and method of using the device for measuring the concentration of intragastric contents, and more particularly to an innovative device and method to measure the concentration of intragastric contents with an electro-chemical specimen and detector.

2. Description of Related Art Including Information Disclosed Under 37 CFR 1.97 and 37 CFR 1.98

Conventionally, the remaining volume of a patient's stomach was measured by adsorbing the stomach with a nasogastric tube. With the nasogastric tube feeding a patient with a large remaining volume of stomach, possible symptoms of the patient or potential hazards arising from inhalation pneumonia must be considered. Up to now, many physicians, nurses and nutritionists still take the volume of stomach remainders as the basis of deciding whether to stop intestinal feeding.

However, this past procedure cannot identify efficiently the extra stomach volume arising from emptying of external feeds or from endogenous excretion. Thus, the way of detecting the stomach's emptying conditions with the volume of stomach remainders is often restrained by the poor sensitivity and failure to fully adsorb the intragastric contents. Moreover, the volume of adsorbed stomach remainders is not sensitive in operation, and it is also impossible to distinguish the residual intestinal composition from numerous endogenous secretions.

In view of the existing problems in monitoring and measuring the stomach emptying conditions as mentioned above, a refractometer has been developed to measure the refraction value of the intragastric food for examining stomach emptying conditions. The refractometer is used to check the concentration of fluids with the light refraction principle, of which the refraction value of foods in the gastric juice is proportional to food concentration. When the food concentration prior to tube feeding is set as 100%, the refraction value is measured with the refractometer. After feeding for a period of time, 2-3 cc gastric juice is extracted to measure the refraction value, and it is converted into the final food concentration. Next, 30 cc of physiological salt solution is injected into the stomach via the nasogastric tube and fully mixed with the gastric juice. Then 2-3 cc of the mixture is extracted to measure the concentration of diluted gastric juice, and finally, the volume of the gastric juice is calculated. However, it is found from actual experience that the typical measuring method for stomach emptying is suitable only provided that the remainders in the stomach are in a liquid state. If solid substances are contained, the light refraction effect will be influenced, thus yielding a possibly bigger error of measurement results of the refractometer in disagreement with the standards.

It is observed that the saliva and intestinal juice from spontaneous secretion is not a clear solution, and some solid substances are contained in the gastrointestinal tract in the human body. So, the typical measurement method may still lead to bigger errors and poorer accuracy, even if the stomach remainders are in a fully liquid state.

Thus, to overcome the aforementioned problems of the prior art, it would be an advancement in the art to provide an improved structure that can significantly improve efficacy.

Therefore, the inventor has provided the present invention of practicability after deliberate design and evaluation based on years of experience in the production, development and design of related products.

### BRIEF SUMMARY OF THE INVENTION

Based on the unique present invention, there a device for measuring the concentration of intragastric content mainly composed of an electrochemical specimen and a detector. It is possible to measure directly the concentrations of saccharide, protein and amino acid in the intragastric content, so that the measurement is not affected by any solid substance, enabling the concentration of intragastric content and stomach emptying conditions to be measured more accurately and conveniently.

Additionally, it is the first time that electrochemical specimen technology has been applied to measure the concentration of intragastric contents; notwithstanding, it is already used in other medical applications (e.g. measurement of blood sugar). It is also found that it can efficiently resolve the problem of the bigger interference readings caused by solids arising from typical refractometer measurement, helping to improve substantially the measurement accuracy of stomach emptying, control the feeding dose of patients and guarantee the recovery process.

Although the invention has been explained in relation to its preferred embodiment, it is to be understood that many other possible modifications and variations can be made without departing from the spirit and scope of the invention as hereinafter claimed.

### BRIEF DESCRIPTION OF THE SEVERAL VIEWS OF THE DRAWINGS

FIG. 1 shows an exploded perspective view of the preferred embodiment of measuring device of the present invention.

FIG. 2 shows an exploded perspective view of the electro-chemical specimen of measuring device of the present invention.

FIG. 3 shows a schematic view of the measurement status of the measuring device of the present invention.

FIG. 4 shows an illustration of a literal block diagram of the measurement steps of the measuring device.

FIG. 5 shows an illustration of a supplementary view of the measurement steps of the measuring device.

FIG. 6 shows another illustration of the supplementary view of the measurement steps of the measuring device.

### DETAILED DESCRIPTION OF THE INVENTION

The features and the advantages of the present invention will be more readily understood upon a thoughtful deliberation of the following detailed description of a preferred embodiment of the present invention with reference to the accompanying drawings.

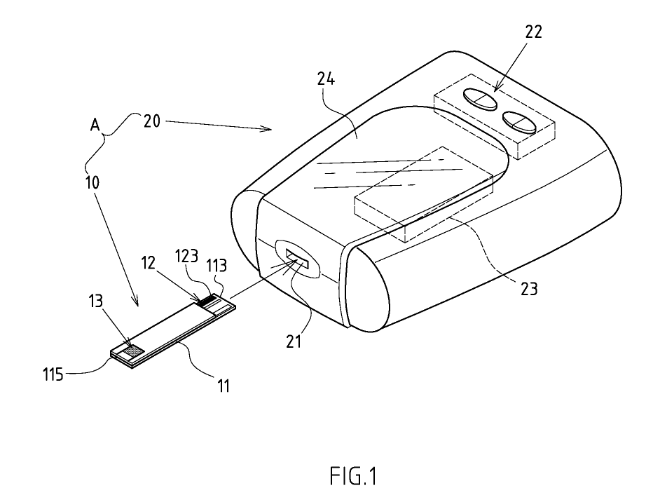

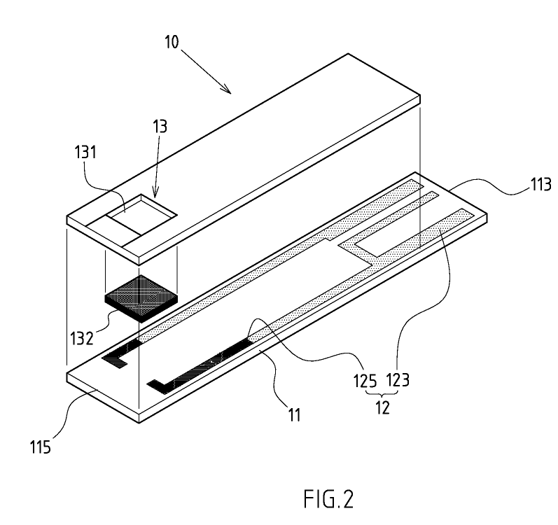

FIGS. 1-2 depict preferred embodiments of device and method of the present invention for measuring the concentration of intragastric contents. The embodiments are provided only for explanatory purposes with respect to the patent claims.

The device for measuring A comprises an electro-chemical specimen 10, containing: a board type insulating substrate 11 with an insertion end 113 and a sensing end 115; an electrode unit 12, containing a detection zone 123 and a reaction zone 125; and a reaction portion 13. The detection zone 123 corresponds to the insertion end 113 of the board type insulating substrate 11, whilst the reaction zone 125 corresponds to the sensing end 115 of the board type insulating substrate 11. The reaction portion 13 is assembled onto the sensing end 115 of the board type insulating substrate 11 and aligned with the reaction zone 125 of the electrode unit 12. Moreover, the reaction portion 13 is provided with an intragastric object inlet 131 and a chemical reaction zone 132.

The device also includes a detector 20, containing: a measurement slot 21, used for mating the insertion end 113 of the electro-chemical specimen 10; a voltage generation unit 22, used to generate a preset voltage; a detection processing unit 23, used to detect the current signals generated by the electro-chemical specimen 10 containing intragastric objects within a time cycle, and interrelate the current signals with the concentration of the intragastric objects; and a measurement display screen 24, used to display the measurement results (i.e. concentration) of the detector 20.

The measuring device A is used to measure the concentrations of saccharide, protein and amino acid in the intragastric objects.

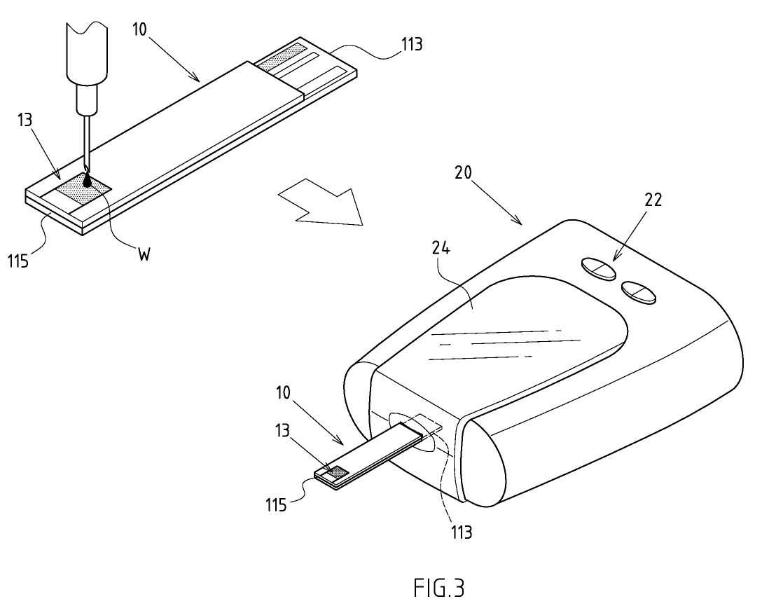

Based on the structures of the aforementioned measuring device A, the intragastric objects are detected and measured as shown in FIG. 3 (in conjunction with FIG. 2), wherein the intragastric objects W are dripped into the reaction portion 13 from the intragastric object inlet 131 of the electro-chemical specimen 10 (or by syringe). The insertion end 113 of the electro-chemical specimen 10 is inserted into the measurement slot 21 of the detector 20, then the voltage generation unit 22 of the detector 20 generates a preset voltage to the electrode unit 12 of the electro-chemical specimen 10. In such a case, the saccharide, protein and amino acid, etc, in the intragastric objects W will yield a chemical reaction with the chemical reaction zone 132 in the reaction portion 13 so as to generate electrons. The electrons will be accumulated in the reaction zone 125 of the electrode unit 12, so the detection processing unit 23 of the detector 20 detects the amount of electrons in the reaction zone 125. The current value is computed by the built-in conversion formula of the detection processing unit 23, making it possible to calculate the concentration of the intragastric objects W and display it on the measurement display screen 24.

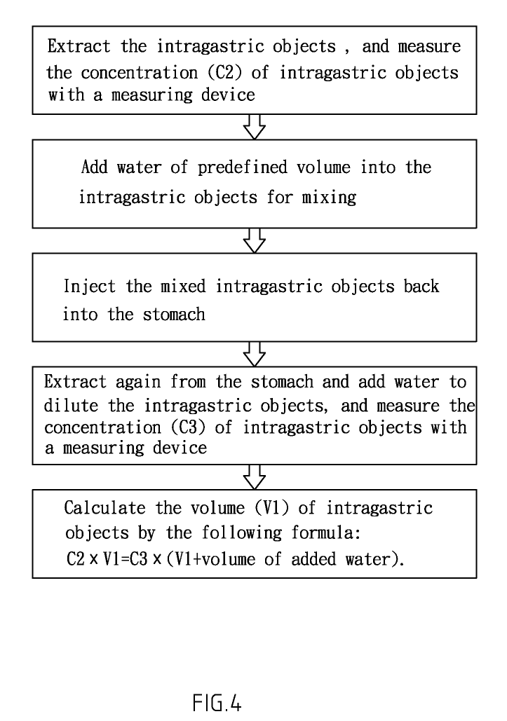

Furthermore, the measurement method of the measuring device may contain the following steps: (referring to FIG. 4)

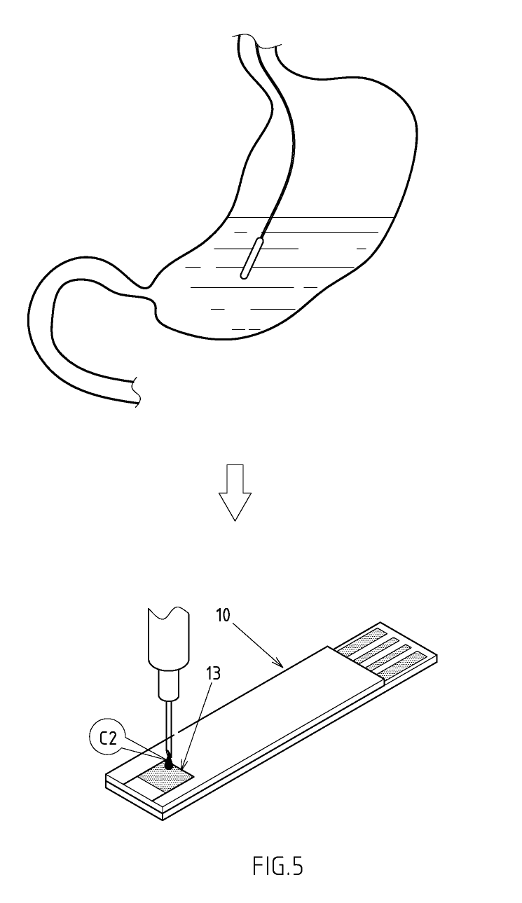

A. Extract the intragastric objects of the testee and measure the concentration (C2) of intragastric objects with a measuring device (in conjunction with FIG. 5);

B. Add water of predefined volume into the intragastric objects for mixing purpose;

C. Inject the mixed intragastric objects back into the stomach;

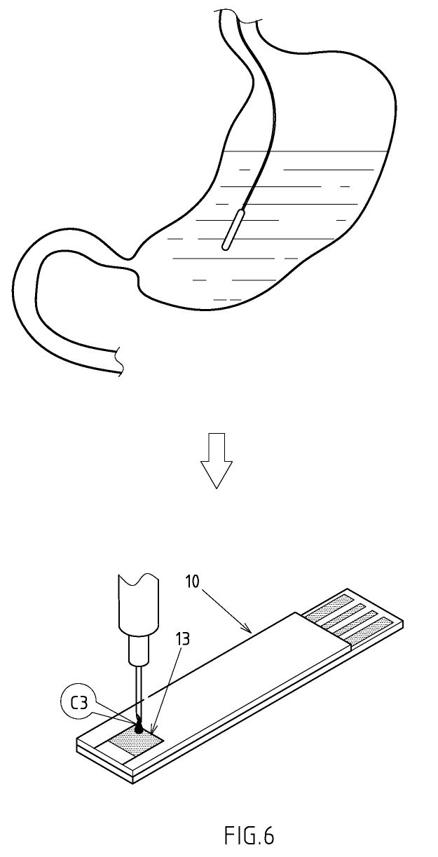

D. Extract again from the stomach and add water to dilute the intragastric objects, then measure the concentration (C3) of intragastric objects with a measuring device (in conjunction with FIG. 6);

E. Calculate the volume (V1) of intragastric objects by the following formula:

C2×V1=C3×(V1+volume of added water).

Before the first step of extracting intragastric objects, the measuring device could be used to measure the concentration(C1) of a predefined solution, such that the concentrations (C2), (C3) measured are a relative value, namely: C2/C1%, C3/C1%: residual concentration of food.

1. A device for measuring concentration of intragastric contents, the device comprising:
an electro-chemical specimen, comprising: a board type insulating substrate with an insertion end and a sensing end; an electrode unit, with a detection zone and a reaction zone, said detection zone corresponding to the insertion end of the board type insulating substrate, said reaction zone corresponding to the sensing end of the board type insulating substrate, and a reaction portion, being assembled onto the sensing end of the board type insulating substrate, and aligned with the reaction zone of the electrode unit, said reaction portion being provided with an intragastric object inlet and a chemical reaction zone; and
a detector, comprising: a measurement slot, mating the insertion end of the electro-chemical specimen; a voltage generation unit, generating a preset voltage; a detection processing means to detect the current signals generated by the electro-chemical specimen containing intragastric objects within a time cycle, and interrelate the current signals with the concentration of the intragastric objects; and a measurement display screen, means to display the measurement results of the detector.

2. The device for measuring defined in claim 1, measuring concentrations of saccharide, protein and amino acid in the intragastric contents.

3. A method of measuring with the device defined in claim 1, the method comprising the following steps:
extracting intragastric content and measuring concentration thereof from a first source with said device;
B. adding water of predefined volume into the intragastric content and mixing;
injecting the mixed intragastric content back to said first source;
extracting water from said first source and adding water to dilute the intragastric contents and measuring concentration of intragastric objects with the measuring device; and
calculating volume (V1) of intragastric contents by the following formula:
C2×V1=C3×(V1+volume of added water).

4. The method of measuring defined in claim 3, wherein the first step of extracting intragastric content is conducted after the measuring device is used to measure the concentration of predefined solution, concentrations of later measurements being measured against a relative value, namely: C2/C1%, C3/C1%: residual concentration of food.
